(19) United States
(12) Patent Application Publication
(10) Pub. No.: US 2016/0278712 A1
(43) Pub. Date: Sep. 29, 2016

(54) Living Body Information Sensor

(71) Applicant: Rohm Co., Ltd., Kyoto (JP)

(72) Inventors: Takeshi Sagara, Kyoto (JP); Koji Saito, Kyoto (JP)

(21) Appl. No.: 15/070,285

(22) Filed: Mar. 15, 2016

(30) Foreign Application Priority Data
Mar. 26, 2015 (JP) 2015064905
Mar. 31, 2015 (JP) 2015071722

(51) Int. Cl. A61B 5/00; A61B 5/1455; A61B 5/024

(52) U.S. Cl. CPC A61B 5/7225; A61B 5/02427; A61B 5/14552

(57) ABSTRACT

The living body information sensor according to the present invention can be attached to a living body. A light emitting unit emits light to the living body. The light receiving unit receives reflected light from the living body that is included in the light emitted by the light emitting unit to the living body, and outputs a signal according to intensity of the reflected light. A control device generates information about the living body based on the signal from the light receiving unit. The control device controls the light emitting unit to turn on and off in an alternate manner. The control device sets a total of a turn-on time of the light emitting unit in a prescribed time interval to be smaller when the living body information sensor is attached to the living body than when the living body information sensor is not attached to the living body.

### BACKGROUND OF THE INVENTION

1. Field of the Invention

The present invention relates to a living body information sensor, and particularly to a wearable living body information sensor.

2. Description of the Background Art

Wearable living body information sensors have been conventionally known. For example, Japanese Patent Laying-Open No. 2012-143316 (PTD 1) discloses a wearable living body information sensor. The living body information sensor includes a first and second optical sensors and an arithmetic circuit. The first optical sensor includes a first light emitting unit emitting light of the first luminescence intensity to a living body, and a first light receiving unit receiving the light emitted from the first light emitting unit and reflected inside the living body to generate a first light receiving signal. The second optical sensor includes a second light emitting unit emitting light of the second luminescence intensity lower than the first luminescence intensity to a living body, and a second light receiving unit receiving the light emitted from the second light emitting unit and reflected inside the living body to generate a second light receiving signal. The arithmetic circuit subtracts the second light receiving signal from the first light receiving signal to obtain pulse wave data.

### SUMMARY OF THE INVENTION

In order to suppress power consumption of the living body information sensor, the light emitting unit may be controlled so as to repeatedly turn on and off in an alternate manner. When the light emitting unit turns on and off at intervals by which a user can recognize that the light emitting unit repeatedly turns on and off, however, the light emitting unit seems to blink, which may cause the user to feel uncomfortable.

On the other hand, when the light emitting unit is turned on and off at relatively shorter intervals to such an extent that the user does not feel uncomfortable, power consumption of the living body information sensor may be unable to be sufficiently suppressed.

Also, when living body information such as pulses or an oxygen saturation concentration in blood is measured while the user wearing a living body information sensor is exercising outdoors or the like, sunlight including infrared light enters the living body information sensor, which may cause measurement errors.

Therefore, an object of the present invention is to provide a living body information sensor capable of suppressing power consumption. Another object of the present invention is to provide a living body information sensor capable of improving measurement accuracy. A living body information sensor according to one embodiment of the present invention can be attached to a living body. A light emitting unit emits light to the living body. A light receiving unit receives reflected light from the living body that is included in the light emitted by the light emitting unit to the living body, and outputs a signal according to intensity of the reflected light. A control device generates information about the living body based on the signal from the light receiving unit. The control device controls the light emitting unit to repeatedly turn on and off in an alternate manner. The control device sets a total of a turn-on time of the light emitting unit in a prescribed time interval to be smaller when the living body information sensor is attached to the living body than when the living body information sensor is not attached to the living body.

The “turn-on time” means a time interval from the time when the light emitting unit having been turned off is turned on to the time when the light emitting unit is turned off for the first time after the time when the light emitting unit is turned on.

By such a configuration, the total time during which a current flows through the light emitting unit is less when the living body information sensor is attached to the living body than when the living body information sensor is not attached to the living body. Consequently, the power consumption of the living body information sensor is less when this sensor is attached to the living body than when this sensor is not attached to the living body. Namely, the living body information sensor according to the present invention allows suppression of power consumption.

Preferably, the control device sets a turn-on interval of the light emitting unit to be longer when the living body information sensor is attached to the living body than when the living body information sensor is not attached to the living body.

The “turn-on interval” means a time interval from the time when the light emitting unit having been turned on is turned off to the time when the light emitting unit is turned on for the first time after the time when the light emitting unit is turned off.

Preferably, the control device sets the turn-on interval of the light emitting unit based on a first signal received from the light receiving unit in a state where the light emitting unit turns on and a second signal received from the light receiving unit in a state where the light emitting unit turns off.

Preferably, the control device calculates a difference obtained by subtracting a level of the second signal from a level of the first signal. The control device sets the turn-on interval at a first turn-on interval when the difference is smaller than a prescribed threshold value. The control device sets the turn-on interval at a second turn-on interval longer than the first turn-on interval when the difference is larger than the prescribed threshold value.

Preferably, the control device controls the light emitting unit to repeatedly turn on and off in an alternate manner in a prescribed turn-on period. The control device sets a ratio of the turn-on time of the light emitting unit to the prescribed turn-on period to be less when the living body information sensor is attached to the living body than when the living body information sensor is not attached to the living body.

The “turn-on period” means a time interval from the time when the light emitting unit having been turned off is turned on, and then the light emitting unit is once turned off, to the time when the light emitting unit is turned on for the first time after the light emitting unit is turned off. Furthermore, the “ratio of the turn-on time of the light emitting unit to the prescribed turn-on period” will be also hereinafter simply referred to as a “turn-on duty ratio”.

Preferably, the living body information sensor further includes a substrate, a first light shielding wall, and a second light shielding wall. On the substrate, the light emitting unit, the light receiving unit and the control device are formed. The first light shielding wall is disposed to surround the light emitting unit along an outer surrounding edge of the substrate. The second light shielding wall partitions space provided by the substrate and the first light shielding wall into space in which the light emitting unit is located and space in which the light receiving unit is located.

A living body information sensor according to another embodiment of the present invention includes a light receiving unit and generates information about a living body based on a signal from the light receiving unit. The light receiving unit includes a first optical sensor converting first light into a first signal. The first optical sensor includes a first photoelectric conversion element and a first color filter. The first photoelectric conversion element converts the first light into the first signal. The first color filter is formed on an optical path leading to the first photoelectric conversion element, and the first light passes through the first color filter. The living body information sensor further includes an infrared light elimination layer formed on the optical path leading to the first photoelectric conversion element.

The color filter is a color resist film containing pigments such as red, green, and blue. The color resist is made using, in addition to resist components, a pigment dispersed resist containing finely-dispersed pigments as coloring materials.

By such a configuration, the light from which infrared light is eliminated by the infrared light elimination layer enters the first optical sensor. Since the first light included in the light having passed through the infrared light elimination layer can pass through the first color filter, most of the light received by the first photoelectric conversion element is the first light. Consequently, the living body information sensor can generate information about a living body with little influence from infrared light. Namely, the living body information sensor according to the present invention can improve the measurement accuracy.

Preferably, the living body information sensor further includes a control device. The control device generates the information about the living body based on the signal from the light receiving unit. The light receiving unit further includes a second optical sensor. The second optical sensor converts second light into a second signal. The second optical sensor includes a second photoelectric conversion element and a second color filter. The second photoelectric conversion element converts the second light into the second signal. The second color filter is formed on an optical path leading to the second photoelectric conversion element, and the second light passes through this second color filter. The infrared light elimination layer is formed also on the optical path leading to the second photoelectric conversion element. The control device receives the first signal and the second signal from the light receiving unit, and generates the information about the living body based on the first signal and the second signal.

Preferably, the light receiving unit further includes a first substrate on which the first optical sensor and the second optical sensor are formed. The infrared light elimination layer is formed on a surface of the first substrate.

The level of the signal can be a voltage value or a current value of an electrical signal, for example.

Preferably, the infrared light elimination layer includes an infrared cut filter. The infrared cut filter covers the first color filter and the second color filter.

The infrared cut filter is a multilayer film that is obtained by stacking a plurality of thin films having different refractive indexes, and that allows light having a wavelength shorter than that of infrared light to pass therethrough. Examples of the plurality of thin films having different refractive indexes can be those obtained by periodically stacking two films including a TiO₂ film and an SiO₂ film by the sputtering method.

Preferably, the infrared light elimination layer includes an infrared cut resin. The infrared cut resin covers the first substrate.

The infrared cut resin is a resin containing a material that absorbs infrared light (for example, light having a wavelength of 750 nm or higher).

Preferably, the infrared light elimination layer includes an infrared cut filter and an infrared cut resin. The infrared cut filter covers the first color filter and the second color filter. The infrared cut resin is stacked on the infrared cut filter, and covers the first substrate.

Preferably, the living body information sensor further includes a light emitting unit, a second substrate, a first light shielding wall, and a second light shielding wall. The light emitting unit emits light including the first light and the second light. The second substrate has the light emitting unit, the light receiving unit and the control device formed thereon. The first light shielding wall is formed to surround the light emitting unit and the light receiving unit along an outer surrounding edge of the second substrate. The second light shielding wall partitions space provided by the second substrate and the first light shielding wall into space in which the light emitting unit is located and space in which the light receiving unit is located. When the living body information sensor is attached to the living body, light included in the light emitted from the light emitting unit and reflected inside the living body enters the light receiving unit.

Preferably, the light emitting unit is a light emitting diode that emits white light.

Preferably, the first light is green light, and the second light is red light.

Preferably, the control device generates the information about the living body based on a difference between a level of the first signal and a level of the second signal.

Preferably, the information about the living body is a heart rate.

The control device generates the information about the living body based on a ratio between a level of the first signal and a level of the second signal.

Preferably, the information about the living body is an oxygen saturation concentration in blood.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a cross-sectional view showing the configuration of a living body information sensor according to the first embodiment.

FIG. 2 is a functional configuration diagram of the living body information sensor in FIG. 1.

FIG. 3A is a time chart illustrating the operation of the living body information sensor according to the first embodiment, which shows waveforms of signals from a G sensor and an R sensor.

FIG. 3B is a time chart illustrating the operation of the living body information sensor according to the first embodiment, which shows a waveform of a signal showing the level difference between the signals from the G sensor and the R sensor.

FIG. 4 is a functional configuration diagram of a control device in FIG. 2.

FIG. 5 is a functional configuration diagram for illustrating details of an arithmetic unit in FIG. 4.

FIG. 6 is a flowchart showing a process performed by a control unit to set a turn-on interval of a light emitting unit.

FIG. 7 is a diagram showing the state where the living body information sensor is not attached to a human body.

FIG. 8 is a time chart showing the manner in which a current flowing through the living body information sensor changes periodically.

FIG. 9A is a diagram showing a frequency spectrum illustrating the operation of a heart rate extraction processing unit, which shows a frequency spectrum of a digital signal generated in an FFT unit in FIG. 4.

FIG. 9B is a diagram showing a frequency spectrum illustrating the operation of the heart rate extraction processing unit, which shows a frequency spectrum of another digital signal generated in the FFT unit in FIG. 4.

FIG. 10A is a diagram showing the manner in which a wrist watch equipped with the living body information sensor is attached to a wrist.

FIG. 10B is a diagram showing the living body information sensor mounted in the wrist watch in FIG. 10A.

FIG. 11 is a cross-sectional view showing the configuration of a living body information sensor according to the second embodiment.

FIG. 12 is a cross-sectional view showing the configuration of a light receiving unit included in the living body information sensor according to the second embodiment.

FIG. 13 is a diagram showing spectral characteristics of an infrared cut filter, a G filter, an R filter, and a photodiode.

FIG. 14 is a cross-sectional view showing the configuration of a light receiving unit included in a living body information sensor according to the first modification of the second embodiment.

FIG. 15 is a cross-sectional view showing the configuration of a light receiving unit included in a living body information sensor according to the second modification of the second embodiment.

FIG. 16 is a functional configuration diagram of a living body information sensor according to the third embodiment.

FIG. 17 is a diagram showing the relation among the wavelength of light, the molar absorption coefficient of hemoglobin, and the spectral sensitivity of the G filter, and the spectral sensitivity of the R filter.

FIG. 18 is a functional configuration diagram of an arithmetic unit included in a living body information sensor according to the third embodiment.

### DESCRIPTION OF THE PREFERRED EMBODIMENTS

Embodiments of the present invention will be hereinafter described in detail with reference to the accompanying drawings, in which the same or corresponding components are denoted by the same reference characters, and a description thereof will not be repeated.

### First Embodiment

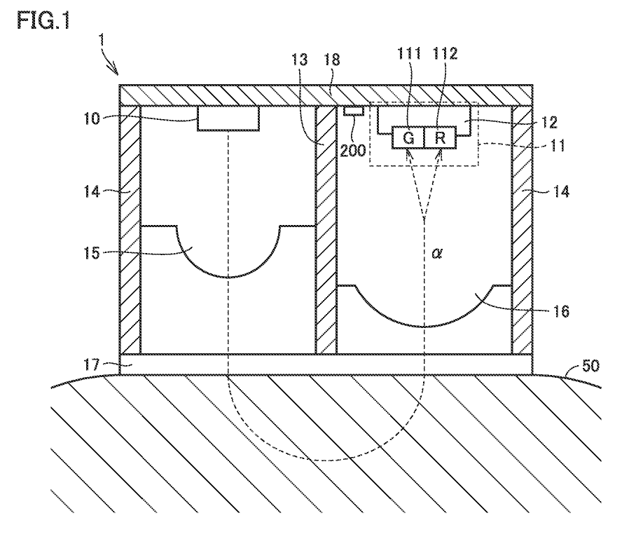

FIG. 1 is a cross-sectional view showing the configuration of a living body information sensor 1 according to the first embodiment. In the first embodiment, an explanation will be given with regard to the case where living body information sensor 1 is used to measure a heart rate as information about a human body.

Referring to FIG. 1, living body information sensor 1 includes a light emitting unit 10 provided as a white LED, a light receiving unit 11, light shielding walls 13 and 14, lenses 15 and 16, a transparent plate 17, a substrate 18, and a control device 200.

Substrate 18 has a surface on which light emitting unit 10, light receiving unit 11 and control device 200 are formed. Light receiving unit 11 includes a silicon substrate 12, and a G (green light) sensor 111 and an R (red light) sensor 112 that are formed on silicon substrate 12. Control device 200 may be formed on silicon substrate 12.

On the edge of the surface of substrate 18, light shielding wall 14 is formed so as to surround light emitting unit 10 and light receiving unit 11. Light shielding wall 14 serves to prevent external light from entering light receiving unit 11. In the center of the surface of substrate 18, light shielding wall 13 is formed for preventing white light, which has been emitted from light emitting unit 10, from directly entering light receiving unit 11. In other words, the space provided by light shielding wall 14 is partitioned by light shielding wall 13 into space in which light emitting unit 10 is located and space in which light receiving unit 11 is located.

Lens 15 is provided in the direction in which light emitting unit 10 emits light and lens 16 is provided in the direction in which light receiving unit 11 receives light. Light shielding walls 13 and 14 have openings that are closed by transparent plate 17. For detecting a heart rate, living body information sensor 1 is arranged such that the surface of transparent plate 17 comes in close contact with the surface of human body 50.

The white light emitted from light emitting unit 10 is applied through lens 15 and transparent plate 17 to human body 50. Light a included in white light and reflected inside the human body passes through transparent plate 17 and lens 15, and enters light receiving unit 11. G sensor 111 receives green light included in light a, and outputs a signal according to the intensity of this green light to control device 200. G sensor 111 receives red light included in light a, and outputs a signal according to the intensity of this red light to control device 200. Control device 200 calculates a heart rate based on the signals from G sensor 111 and R sensor 112, and generates a signal showing the heart rate.

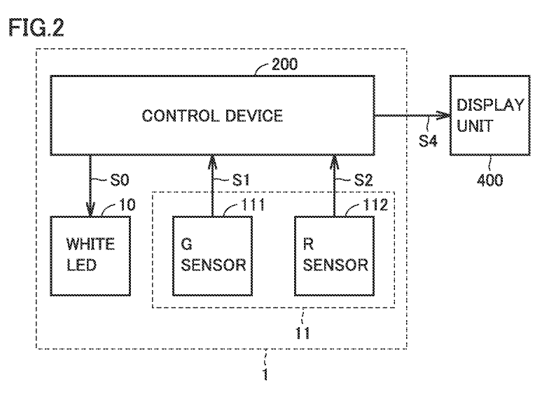

The functional configuration of living body information sensor 1 will be hereinafter described. FIG. 2 is a functional configuration diagram of living body information sensor 1. In response to an instruction from a user to start measurement, control device 200 outputs a control signal S0 to light emitting unit 10. In response to control signal S0 having reached a prescribed level, light emitting unit 10 emits light and outputs white light to a human body. The white light output from light emitting unit 10 is reflected inside the human body, and this reflected light enters G sensor 111 and R sensor 112. Since a part of the white light is absorbed into skin, blood and the like at this time, the intensity of the reflected light changes in accordance with the pulse wave and body motion of the human body. Specifically, the intensity of the green light included in the reflected light changes in accordance with the pulse wave and the body motion of the human body. The intensity of the red light included in the reflected light changes in accordance with the human body motion, but hardly changes in accordance with pulsation in the human body.

G sensor 111 receives light emitted from light emitting unit 10 and reflected inside the human body, and generates a signal S1 according to the intensity of the green light included in the received light. The level of signal S1 (for example, a voltage) increases as the light intensity of the incoming green light increases. As described above, the level of signal S1 changes in accordance with the pulsation and body motion of the human body.

R sensor 112 receives light emitted from light emitting unit 10 and reflected inside the human body, and generates a signal S2 according to the intensity of the red light included in the received light. The level of signal S2 (for example, a voltage) increases as the light intensity of the incoming red light increases. As described above, signal S2 changes in accordance with human body motion, but hardly changes in accordance with pulsation of the human body.

Control device 200 may amplify at least one of signals S1 and S2 such that the amplitudes of the body motion components of signals S1 and S2 that change in the same period are approximately the same. Control device 200 calculates a signal that shows the level difference between signal S1 and a signal obtained by amplifying signal S2, and then, obtains a pulse wave in a human body based on the signal showing this level difference.

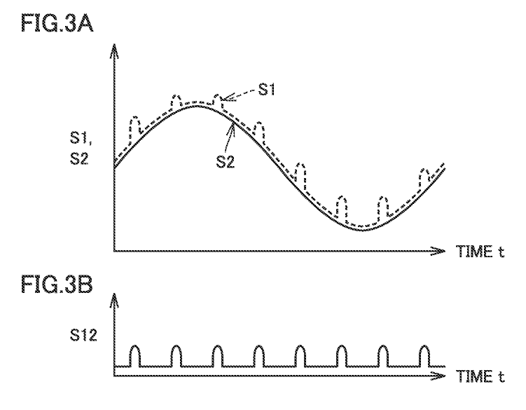

FIGS. 3A and 3B are time charts each illustrating the operation of living body information sensor 1. FIG. 3A illustrates waveforms of signals S1 and S2 while FIG. 3B illustrates a waveform of a signal S12 that shows the level difference between signal S1 and signal S2. In FIGS. 3A and 3B, signal S1 includes a body motion component that changes in a relatively long period and in a relatively large amplitude, and a pulse wave component that changes in a relatively short period and in a relatively small amplitude. Also, signal S2 includes a body motion component that is approximately the same as that of signal S1. Signal S12 includes only a pulse wave component.

Again referring to FIG. 2, control device 200 counts the pulse number per minute starting from signal S12 to thereby calculate a heart rate. Then, control device 200 outputs a signal S4 showing this heart rate to a monitor (a display unit 400) of a wrist watch-type wearable device, for example. Display unit 400 causes its screen to display characters, images and the like showing a heart rate according to signal S4 from control device 200. Control device 200 and display unit 400 may communicate with each other through wired connection or wirelessly.

In order to suppress power consumption of living body information sensor 1, light emitting unit 10 may be controlled so as to repeatedly turn on and off. If the turn-on interval of light emitting unit 10 in living body information sensor 1 is relatively long, this light emitting unit 10 seems to blink, which may cause the user to feel uncomfortable.

In this case, when living body information sensor 1 is attached to a human body, living body information sensor 1 comes in close contact with the human body in the state where light emitting unit 10 faces the human body. Accordingly, the user usually cannot see that light emitting unit 10 turns on and off. In other words, in the case where living body information sensor 1 is attached to a living body, it is not necessary to suppress blinking of light emitting unit 10. Thus, the turn-on interval of light emitting unit 10 can be lengthened to such an extent as not to cause any problems in heart rate measurement. The turn-on interval of light emitting unit 10 is lengthened, which causes a decrease in total time during which a current flows through the light emitting unit in a prescribed time interval. Consequently, power consumption of the living body information sensor can be suppressed.

Thus, in the first embodiment, an explanation will be given with regard to the configuration for suppressing power consumption of living body information sensor 1 by controlling light emitting unit 10 to turn on and off such that the total turn-on time of light emitting unit 10 in a prescribed time interval is less when living body information sensor 1 is attached to the human body than when living body information sensor 1 is not attached to the human body.

The functional configuration of control device 200 will be first hereinafter described. The following description includes an explanation about characteristic changes of the turn-on interval of light emitting unit 10 in the first embodiment.

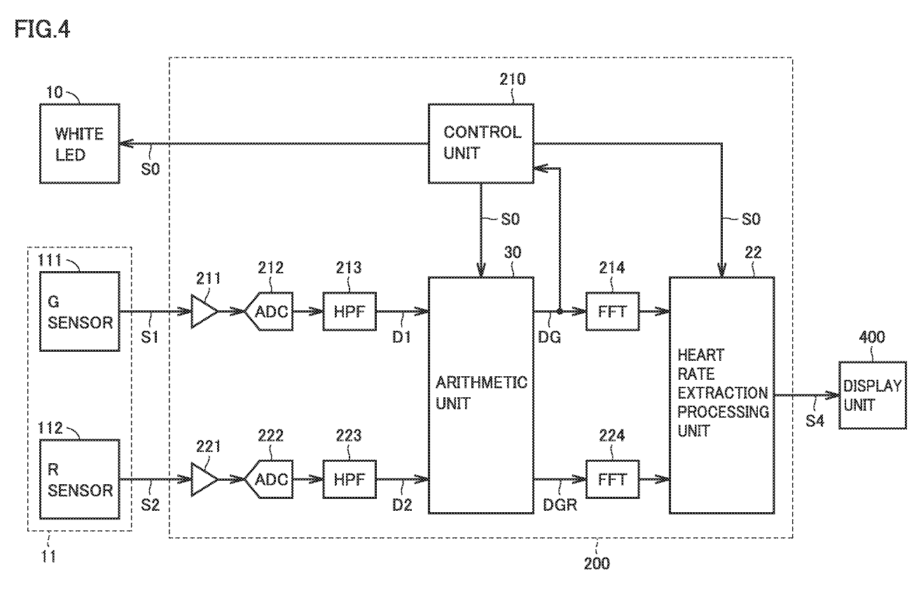

FIG. 4 is a functional configuration diagram of control device 200. Referring to FIG. 4, control device 200 includes amplifiers 211 and 221, AD (analog-to-digital) converters 212 and 222, high-pass filters (HPF) 213 and 223, an arithmetic unit 30, FFT (Fast Fourier Transform) units 214 and 224, a heart rate extraction processing unit 22, and a control unit 210.

Control unit 210 turns control signal S0 into an “H” level and an “L” level in an alternate manner at a frequency sufficiently higher than the frequency of the pulse wave, to cause light emitting unit 10 to turn on and off in a prescribed period, and also controls the entire control device 200 in synchronization with control signal SO.

Control unit 210 sets the turn-on interval of light emitting unit 10 based on the signal from light receiving unit 11. This setting process performed by control unit 210 will be described later in detail.

G sensor 111 receives light emitted from light emitting unit 10 and reflected inside the human body, and the light leaked thereinto from the outside in accordance with the body motion. Then, G sensor 111 outputs signal S1 of the level in accordance with the light intensity of the green light included in the received light. R sensor 112 receives light emitted from light emitting unit 10 and reflected inside the human body, and the light leaked thereinto from the outside in accordance with the body motion. Then, R sensor 112 outputs signal S2 of the level in accordance with the light intensity of the red light included in the received light.

Amplifier 211 amplifies signal S1 output from G sensor 111. AD converter 212 converts the output signal from amplifier 211 into a digital signal. High-pass filter 213 removes a direct-current (DC) component in the output signal of AD converter 212. The signal having passed through high-pass filter 213 is output to arithmetic unit 30 as a digital signal D1. Amplifier 221 amplifies signal S2 output from R sensor 112. AD converter 222 converts the output signal from amplifier 221 into a digital signal. High-pass filter 223 removes a DC component in the output signal of AD converter 222. The signal having passed through high-pass filter 223 is output to arithmetic unit 30 as a digital signal D2.

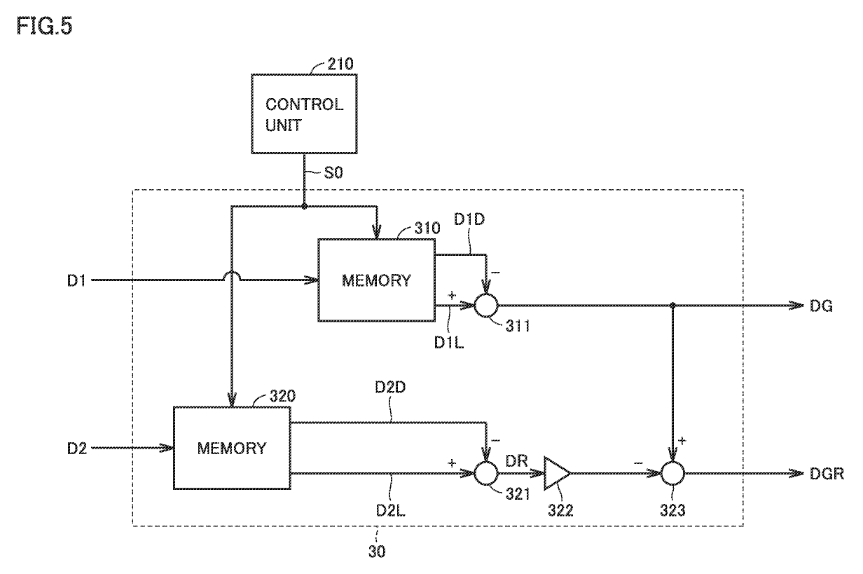

Arithmetic unit 30 includes memories 310 and 320, subtractors 311, 321 and 323, and an amplifier 322, as shown in FIG. 5. Memory 310 temporarily stores digital signal D1 as a digital signal D1D in a time zone during which control signal S0 is at an “L” level (that is, the time zone during which light emitting unit 10 is turned off), and temporarily stores digital signal D1 as a digital signal D1L in a time zone during which control signal S0 is at an “H” level (that is, the time zone during which light emitting unit 10 is turned on). After that, memory 310 outputs digital signals D1D and D1L to subtracter 311.

Digital signal D1D includes a noise component resulting from the light and the like having leaked into G sensor 111 from the outside in the time zone during which light emitting unit 10 is turned off. This noise component is included also in digital signal D1L. Subtracter 311 subtracts digital signal D1D from digital signal D1L, and outputs a digital signal DG. Therefore, digital signal DG is a signal obtained by removing a noise component from digital signal D1L.

Similarly, memory 320 temporarily stores digital signal D2 as a digital signal D2D in a time zone during which control signal S0 is at an “L” level (that is, the time zone during which light emitting unit 10 is turned off), and also temporarily stores digital signal D2 as a digital signal D2L in a time zone during which control signal S0 is at an “H” level (that is, the time zone during which light emitting unit 10 is turned on). After that, memory 320 outputs digital signals D2D and D2L to subtracter 321.

Digital signal D2D includes a noise component resulting from the light and the like having leaked into R sensor 112 from the outside in the time zone during which light emitting unit 10 is turned off. This noise component is included also in digital signal D2L. Subtracter 321 subtracts digital signal D2D from digital signal D2L, and outputs digital signal DR. Therefore, digital signal DR is a signal obtained by removing a noise component from digital signal D2L.

Digital signal DG mainly includes a body motion component and a pulse wave component. Digital signal DR mainly includes a body motion component. Amplifier 322 amplifies digital signal DR such that the level of the body motion component included in digital signal DG and the level of the body motion component included in digital signal DR are almost the same. Subtracter 323 subtracts digital signal DR from digital signal DG, and outputs a digital signal DGR. Digital signal DGR is a signal obtained by subtracting digital signal DR including a body motion component from digital signal DG including a body motion component and a pulse wave component. Accordingly, it can be said that this digital signal DGR mainly includes a pulse wave component.

In addition, although digital signal DR is amplified by amplifier 322 in arithmetic unit 30 shown in FIG. 5, the present invention is not limited thereto, but at least one of digital signals DG and DR may be amplified or attenuated such that the levels of the body motion components included in digital signals DG and DR are almost the same.

Again referring to FIG. 4, FFT unit 214 applies fast Fourier transform to digital signal DG to generate a frequency spectrum of digital signal DG, and outputs the generated frequency spectrum to heart rate extraction processing unit 22. FFT unit 224 applies fast Fourier transform to digital signal DGR to generate a frequency spectrum of digital signal DGR, and outputs the generated frequency spectrum to heart rate extraction processing unit 22. Heart rate extraction processing unit 22 calculates the heart rate of a human body based on the frequency spectra output from FFT units 214 and 224, and outputs a signal S4 showing the calculated heart rate to display unit 400. The details of the process performed by heart rate extraction processing unit 22 will be described later.

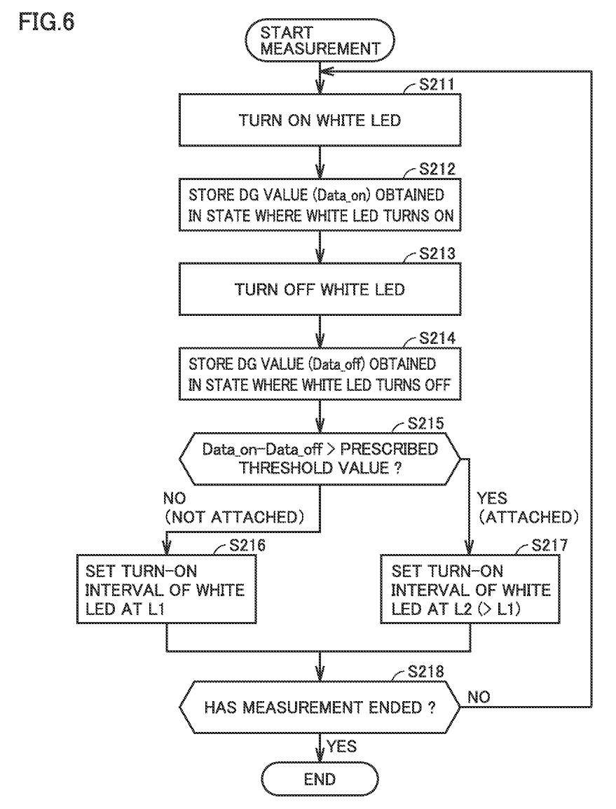

The following is an explanation about the flow of the process performed by control unit 210 to set the turn-on interval of light emitting unit 10 with reference to FIG. 6. Referring to FIG. 6, when the user gives a measurement start instruction to cause living body information sensor 1 to start measurement of the heart rate, control unit 210 turns on light emitting unit 10 in step S211, and advances the process to step S212. In step S212, control unit 210 stores a DG value (Data_on) obtained in the state where light emitting unit 10 turns on, and then, advances the process to step S213. Control unit 210 turns off light emitting unit 10 in step S213, and advances the process to step S214. In step S214, control unit 210 stores a DG value (Data_off) obtained in the state where light emitting unit 10 turns off, and then, advances the process to step S215.

In step S215, control unit 210 determines whether the value obtained by subtracting Data_off from Data_on is greater than a prescribed threshold value. This determination corresponds to a determination as to whether living body information sensor 1 is attached to a human body or not.

When living body information sensor 1 is attached to a human body (see FIG. 1), in the state where light emitting unit 10 turns on, white light is applied to human body 50, and light α reflected inside human body 50 enters light receiving unit 11. The value of digital signal DG (Data_on) is determined according to the intensity of red light included in light α. In the state where light emitting unit 10 turns off, light α does not enter light receiving unit 11. Accordingly, the value of digital signal DG (Data_off) is smaller than Data_on.

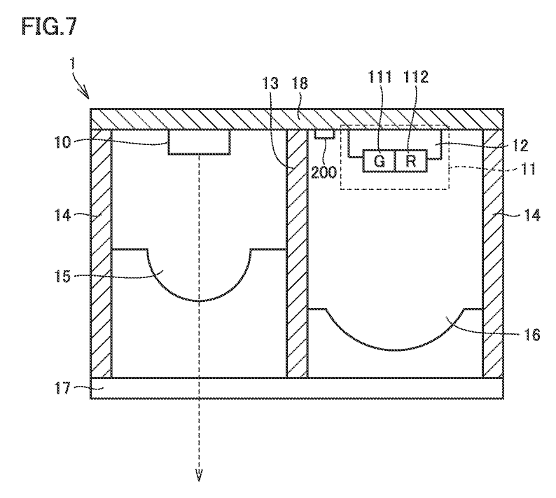

When living body information sensor 1 is not attached to a human body (see FIG. 7), in the state where light emitting unit 10 turns on, the light output from light emitting unit 10 does not reflect inside the human body to enter light receiving unit 11. The same also applies to the state where light emitting unit 10 turns off. In other words, when living body information sensor 1 is not attached to a human body, Data_on and Data_off are almost the same.

Therefore, as a result of comparing Data_on and Data_off, it can be determined that living body information sensor 1 is attached to a human body if both of the values are considerably different from each other, and it can be determined that living body information sensor 1 is not attached to a human body if both of the values are almost the same.

In the first embodiment, it is determined based on the difference between Data_on and Data_off whether living body information sensor 1 is attached to a human body or not. However, it may be determined based on the ratio between Data_on and Data_off whether living body information sensor 1 is attached to a human body or not.

Again referring to FIG. 6, when the value obtained by subtracting Data_off from Data_on is equal to or less than a prescribed threshold value (NO in S215), control unit 210 advances the process to step S216. In step S216, control unit 210 sets the turn-on interval of light emitting unit 10 at a turn-on interval L1, and then, advances the process to step S218. When the value obtained by subtracting Data_off from Data_on is greater than the prescribed threshold value (YES in S215), control unit 210 advances the process to step S217. In step S217, control unit 210 sets the turn-on interval of light emitting unit 10 at a turn-on interval L2 longer than turn-on interval L1, and then, advances the process to step S218.

In step S218, control unit 210 determines whether the heart rate measurement has ended or not in response to the instruction from the user to end the measurement. When the heart rate measurement has ended (YES in S218), control unit 210 ends the process. When the heart rate measurement has not ended (NO in S218), control unit 210 returns the process to step S211.

In order to start heart rate measurement immediately after living body information sensor 1 is attached to a human body, control unit 210 repeatedly performs the process in step S211 to step S218 even if living body information sensor 1 is not attached to a human body.

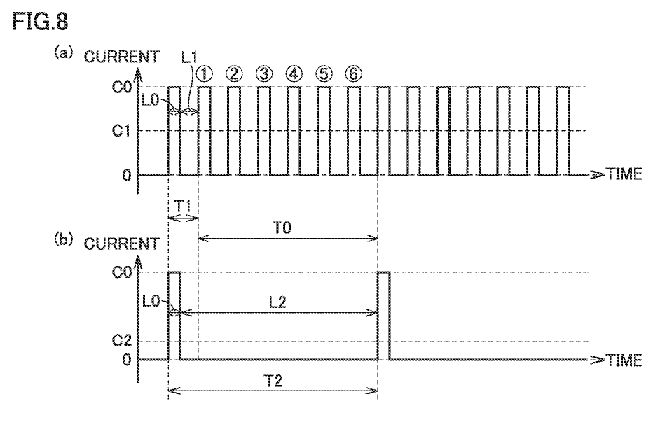

FIG. 8 is a diagram illustrating the difference about power consumption, which is caused by the above-described process performed by control unit 210, between the case where living body information sensor 1 is not attached to a human body and the case where living body information sensor 1 is attached to a human body, which shows a time chart illustrating the manner in which a current flowing through light emitting unit 10 periodically changes. Light emitting unit 10 turns on when the current of a current value C0 flows, and turns off when the current value is 0. A current flows through light emitting unit 10 during turn-on time L0 at current value C0 in the turn-on period. In other words, light emitting unit 10 keeps turning on during turn-on time L0 in the turn-on period, and repeatedly turns on and off periodically according to the periodical change of the current flowing therethrough. Current value C0 and turn-on time L0 are set to be almost constant irrespective of whether living body information sensor 1 is attached to a human body or not.

FIG. 8(a) is a diagram showing the state where living body information sensor 1 is not attached to a human body. In this case, control unit 210 sets the turn-on interval of light emitting unit 10 at turn-on interval L1. As a result, the turn-on period is set at a turn-on period T1 (for example, about 8 ms).

FIG. 8(b) is a diagram showing the state where living body information sensor 1 is attached to a human body. In this case, control unit 210 sets the turn-on interval of light emitting unit 10 at a turn-on interval L2 longer than turn-on interval L1. As a result, turn-on time L0 becomes almost constant, so that turn-on period T2 is longer than turn-on period T1. Turn-on period T2 is about 50 ms to 100 ms, for example.

Referring to FIG. 8(a), when living body information sensor 1 is not attached to a human body, light emitting unit 10 turns on six times during time interval T0, in which case the total turn-on time is 6 LT. On the other hand, referring to FIG. 8(b), when living body information sensor 1 is attached to a human body, light emitting unit 10 does not turn on in time interval T0. Accordingly, the total turn-on time in this case is 0. Specifically, the total time during which a current flows through light emitting unit 10 is less when living body information sensor 1 is attached to a human body than when living body information sensor 1 is not attached to a human body. Therefore, the power consumption of living body information sensor 1 is less when this living body information sensor 1 is attached to a human body than when living body information sensor 1 is not attached to a human body.

In addition, light emitting unit 10 turns on six times during time interval T0 in FIG. 8(a), but does not turn on during time interval T0 in FIG. 8 (b). This is merely by way of example for the sake of explanation, and the turning-on number of light emitting unit 10 in a prescribed time interval is not limited to six or zero.

Focusing on the turn-on duty ratio, a turn-on duty ratio R1 in the case where living body information sensor 1 is not attached to a human body is L0/T1. A turn-on duty ratio R2 in the case where living body information sensor 1 is attached to a human body is L0/T2. Since turn-on period T2 is greater than turn-on period T1, turn-on duty ratio R2 is less than turn-on duty ratio R1. Namely, it can be said that control unit 210 sets the turn-on interval of light emitting unit 10 such that turn-on duty ratio R2 obtained in the case where living body information sensor 1 is attached to a human body is less than turn-on duty ratio R1 obtained in the case where living body information sensor 1 is not attached to a human body. As a result, an average current value C2 obtained in the case where living body information sensor 1 is attached to a human body is less than an average current value C1 obtained in the case where living body information sensor 1 is not attached to a human body. Therefore, power consumption of living body information sensor 1 can be set to be smaller when living body information sensor 1 is attached to a human body than when living body information sensor 1 is not attached to a human body.

In the first embodiment, the turn-on interval of light emitting unit 10 is lengthened, so that the turn-on duty ratio is set to be smaller when living body information sensor 1 is attached to a human body than when living body information sensor 1 is not attached to a human body. However, the method of reducing the turn-on duty ratio is not limited to a method of lengthening the turn-on interval of light emitting unit 10. For example, the turn-on time of light emitting unit 10 in the turn-on period may be shortened when living body information sensor 1 is attached to a human body. Furthermore, when living body information sensor 1 is attached to a human body, the turn-on interval of light emitting unit 10 may be lengthened and the turn-on time of light emitting unit 10 in the turn-on period may be shortened.

From the above description, according to living body information sensor 1 in the first embodiment, power consumption can be suppressed by controlling light emitting unit 10 to turn on such that the total turn-on time of light emitting unit 10 in a prescribed time interval is less when living body information sensor 1 is attached to a human body than when living body information sensor 1 is not attached to a human body.

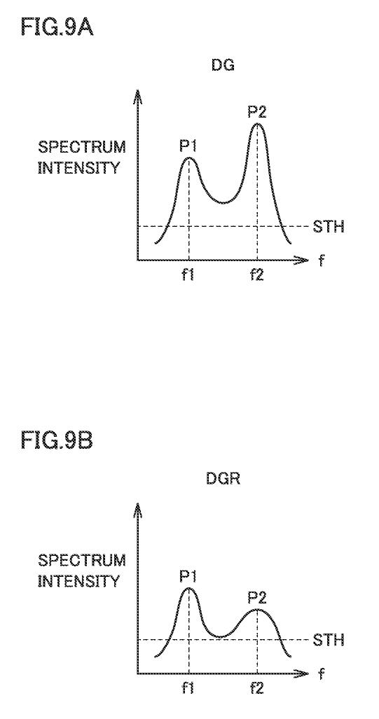

The operation of heart rate extraction processing unit 22 will be hereinafter described with reference to FIGS. 9A and 9B. FIG. 9A is a diagram showing a frequency spectrum of digital signal DG generated in FFT unit 214 (see FIG. 4). FIG. 9B is a diagram showing a frequency spectrum of digital signal DGR generated in FFT unit 224 (see FIG. 4). The frequency of the peak at which the spectrum intensity falls within a range equal to or greater than a predetermined threshold value STH is defined as a candidate for the pulse wave frequency.

When the human body motion is not negligible (for example, when the user is jogging), the frequency spectra of digital signals DG and DGR each show a curved line including two peaks P1 and P2 each protruding upward, as shown in FIGS. 9A and 9B. As described above, digital signal DG mainly includes a body motion component and a pulse wave component while digital signal DGR mainly includes a pulse wave component. In other words, it can be said that digital signal DGR is less in body motion component than digital signal DG. Thus, heart rate extraction processing unit 22 calculates a rate at which the heights of peaks P1 and P2 of the frequency spectrum of digital signal DGR decreases with respect to the heights of peaks P1 and P2 of the frequency spectrum of digital signal DG (the decrease rate). Heart rate extraction processing unit 22 determines a frequency f2 of the peak with the higher decrease rate (in this case, P2) as a frequency of the body motion, and determines frequency f1 of the peak with the lower decrease rate (in this case, P1) as a frequency of the pulse wave. Then, heart rate extraction processing unit 22 calculates a heart rate [times/minute] based on this frequency f1, and outputs signal S4 showing the heart rate [times/minute] to display unit 400.

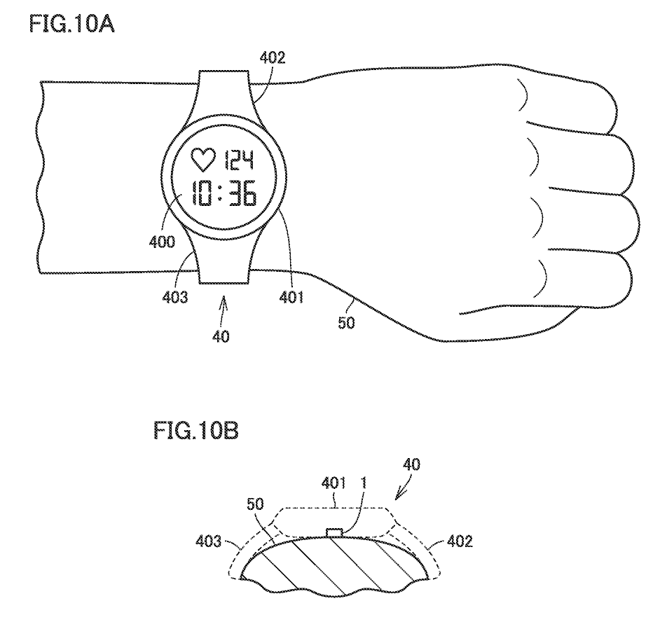

FIG. 10A is a diagram showing the manner in which a wrist watch 40 equipped with living body information sensor 1 is attached to a wrist. Referring to FIG. 10A, wrist watch 40 includes a display unit 400, a wrist watch case 401, and watch bands 402 and 403. Display unit 400 displays a heart rate together with time. FIG. 10B is a diagram showing living body information sensor 1 mounted in wrist watch 40. Referring to FIG. 10B, wrist watch 40 has wrist watch case 401 in which living body information sensor 1 is provided. Living body information sensor 1 is arranged in the bottom surface portion of wrist watch 40. When wrist watch 40 is attached to a wrist, living body information sensor 1 measures the user's heart rate while coming in close contact with the skin surface, and then, outputs a signal showing a heart rate to display unit 400.

The device on which living body information sensor 1 can be mounted is not limited to a wrist watch. Examples of the device on which living body information sensor 1 can be mounted can be glasses, an earphone, or a smart phone.

In the first embodiment, it is determined based on the signal from light receiving unit 11 whether living body information sensor 1 is attached to a human body or not, but the method of determining whether living body information sensor 1 is attached to a human body or not is not limited to the above-described method. For example, this determination may be made based on the signal from the sensor capable of detecting contact between a human body and living body information sensor 1 such as a pressure sensor or a temperature sensor. Alternatively, this determination may be made using a mechanical mechanism like a micro-switch.

Although a heart rate is measured as information about a living body in the first embodiment, the information about the living body is not limited to a heart rate, but may be an oxygen saturation concentration in blood, for example.

According to the living body information sensor in the first embodiment, it goes without saying that not only a heart rate in a human body but also a heart rate in a living body including humans and animals can be detected.

### Second Embodiment

In the first embodiment, the configuration allowing suppression of power consumption has been described in detail. In the second embodiment, the configuration allowing improvement in measurement accuracy will be described in detail.

The structure of light receiving unit 11 will be described in detail in the second embodiment. Since other structures are the same as those in the first embodiment, the description thereof will not be repeated.

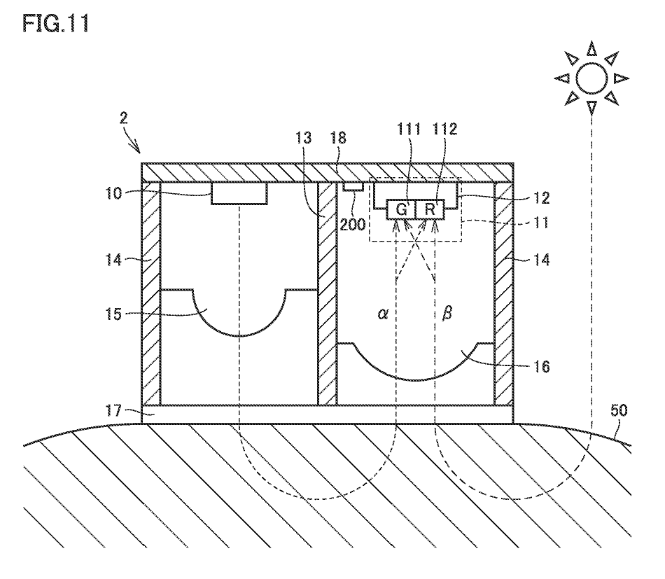

FIG. 11 is a cross-sectional view showing the configuration of a living body information sensor 2 according to the second embodiment.

When a user measures his/her heart rate outdoors or the like while attaching living body information sensor 2 to user's human body 50, light β included in sunlight and having passed through human body 50 or having been reflected inside human body 50 may enter light receiving unit 11. Light β originating from sunlight includes infrared light. G sensor 111 and R sensor 112 exhibit the highest sensitivities to green light and red light, respectively, but the sensitivity to infrared light exhibited by these sensors is not necessarily zero. Accordingly, when living body information sensor 2 measures a heart rate, light β turns into a noise, which may cause errors to occur in the measured heart rate.

Thus, in the second embodiment, an infrared light elimination layer including an infrared cut filter is used to eliminate such infrared light.

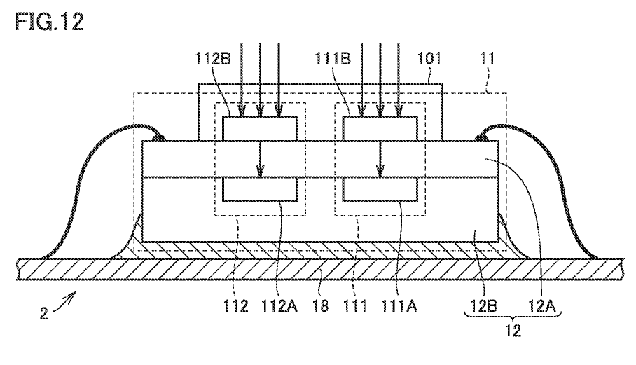

FIG. 12 is a cross-sectional view showing the configuration of light receiving unit 11 included in living body information sensor 2 according to the second embodiment. Referring to FIG. 12, light receiving unit 11 includes a silicon substrate 12, a G sensor 111, an R sensor 112, and an infrared cut filter 101.

G sensor 111 has a photodiode 111A and a G filter 111B. R sensor 112 has a photodiode 112A and an R filter 112B. G filter 111B allows green light to pass therethrough. Photodiode 111A converts the green light having passed through G filter 111B into a signal S1. R filter 112B allows red light to pass therethrogh. Photodiode 112A converts the red light having passed through R filter 112B into a signal S2.

Silicon substrate 12 is divided into an upper layer 12A and a lower layer 12B. Upper layer 12A has a surface on which G filter 111B and R filter 112B are formed. On the boundary between upper layer 12A and lower layer 12B, photodiodes 111A and 112A are provided on the lower layer 12B side. G filter 111B and R filter 112B face photodiodes 111A and 112A, respectively. Accordingly, the light that is to enter photodiode 111A passes through G filter 111B while the light that is to enter photodiode 112A passes through R filter 112B. In other words, G filter 111B is formed on an optical path leading to photodiode 111A, and R filter 112B is formed on an optical path leading to photodiode 112A.

Infrared cut filter 101 is formed on the surface of upper layer 12A, and covers G filter 111B and R filter 112B. Accordingly, the light that is to enter each of photodiode 111A and photodiode 112A passes through the infrared cut filter. In other words, infrared cut filter 101 is formed on an optical path leading to photodiode 111A and also formed on an optical path leading to photodiode 112A.

By such a configuration, most of the light input into photodiode 111A turns into green light while most of the light input into photodiode 112A turns into red light. Such results are due to a difference among spectral characteristics of infrared cut filter 101, G filter 111B and R filter 112B. Focusing on the spectral characteristics in the following description, an explanation will be given with regard to the feature that most of the light input into photodiodes 111A and 112A turn into green light and red light, respectively.

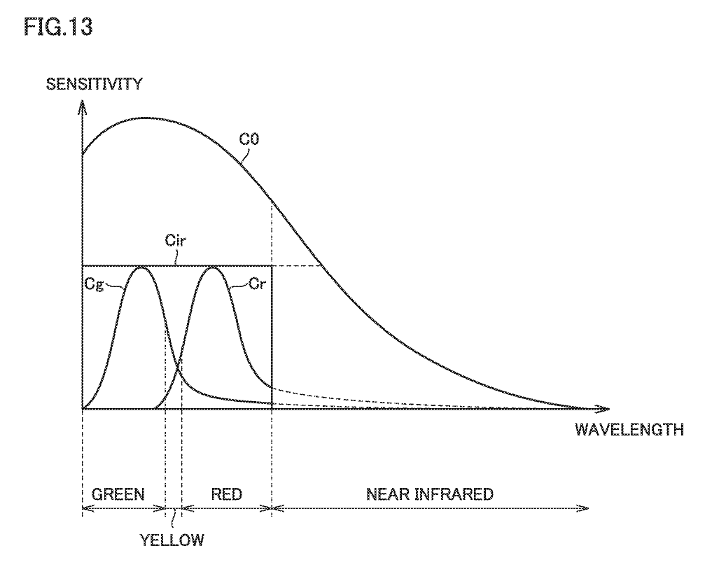

FIG. 13 is a diagram showing spectral characteristics of infrared cut filter 101, G filter 111B, R filter 112B, and photodiode 111A (112A). In FIG. 12, Cir shows the spectral characteristics of infrared cut filter 101; Cg shows the spectral characteristics of G filter 111B; Cr shows the spectral characteristics of R filter 112B; and CO shows the spectral characteristics of photodiode 111A (112A).

When light enters light receiving unit 11, this light first passes through infrared cut filter 101. Referring to FIG. 12, since the sensitivity in the near infrared region of infrared cut filter 101 is approximately 0, the infrared light included in this light cannot pass through infrared cut filter 101. The light having passed through infrared cut filter 101 passes through G filter 111B or R filter 112B. G filter 111B exhibits the highest sensitivity in a green region, so that it allows green light to pass therethrough. R filter 112B exhibits the highest sensitivity in a red region, so that it allows red light to pass therethrough. Consequently, most of the light input into photodiode 111A turns into green light while most of the light input into photodiode 112A turns into red light.

As described above, according to living body information sensor 2 in the second embodiment, the infrared light included in the light entering light receiving unit 11 is eliminated by the infrared light elimination layer including infrared cut filter 101, so that the measurement accuracy for heart rate measurement can be improved.

### First Modification of Second Embodiment

In the second embodiment, an explanation has been given with regard to the case where an infrared light elimination layer includes an infrared cut filter. The infrared light elimination layer does not necessarily have to include an infrared cut filter. In the first modification of the second embodiment, an explanation will be given with regard to the case where an infrared light elimination layer includes an infrared cut resin.

The first modification of the second embodiment is different from the second embodiment only in that the infrared light elimination layer includes an infrared cut resin in place of an infrared cut filter. Since the first modification of the second embodiment is the same as the second embodiment other than this difference, the description thereof will not be repeated.

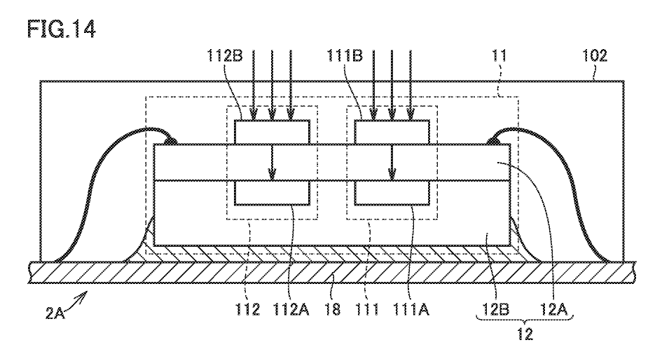

FIG. 14 is a cross-sectional view showing the configuration of a light receiving unit 11 included in a living body information sensor 2A according to the first modification of the second embodiment. Referring to FIG. 14, silicon substrate 12 is entirely covered by an infrared cut resin 102. The spectral characteristics of infrared cut resin 102 are almost the same as those of the infrared cut filter. Therefore, as in the second embodiment, the infrared light included in the light having entered light receiving unit 11 cannot pass through the infrared cut resin. Thus, most of the light input into photodiode 111A turns into green light while most of the light input into photodiode 112A turns into red light.

As described above, according to living body information sensor 2A in the first modification of the second embodiment, the infrared light included in the light entering light receiving unit 11 is eliminated by the infrared light elimination layer including infrared cut resin 102, so that the measurement accuracy for heart rate measurement can be improved.

### Second Modification of Second Embodiment

In the second embodiment, an explanation has been given with regard to the case where the infrared light elimination layer includes an infrared cut filter. In the first modification of the second embodiment, an explanation has been given with regard to the case where the infrared light elimination layer includes an infrared cut resin. Specifically, in each of the second embodiment and the first modification of the second embodiment, an explanation has been given with regard to the case where a single infrared light elimination layer is provided. The number of infrared light elimination layers, however, does not necessarily have to be one. In the second modification of the second embodiment, an explanation will be given with regard to the case where the infrared light elimination layer includes an infrared cut filter and an infrared cut resin so as to consist of two layers.

The second modification of the second embodiment is different from the second embodiment in that the infrared light elimination layer includes an infrared cut resin in addition to an infrared cut filter. Since the second modification of the second embodiment is the same as the second embodiment other than this difference, the description thereof will not be repeated.

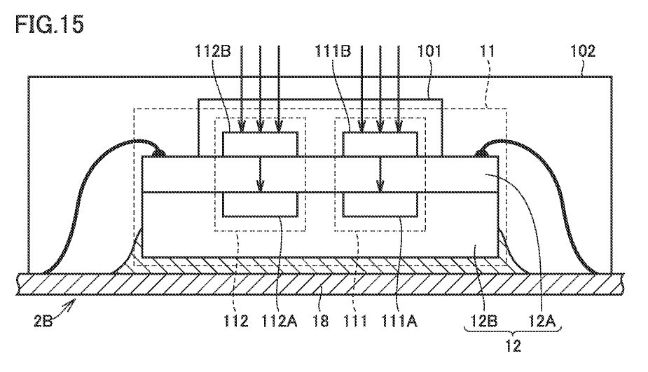

FIG. 15 is a cross-sectional view showing the configuration of a light receiving unit 11 included in a living body information sensor 2B according to the second modification of the second embodiment. Referring to FIG. 15, also in the second modification of the second embodiment, infrared cut filter 101 is formed on the surface of upper layer 12A of silicon substrate 12, and covers G filter 111B and R filter 112B, as in the second embodiment. Furthermore, in the second modification of the second embodiment, infrared cut resin 102 is stacked on infrared cut filter 101. Infrared cut resin 102 covers the entire silicon substrate 12.

By such a configuration, the infrared light included in the light having entered light receiving unit 11 cannot pass through the infrared cut resin and the infrared cut filter. Thus, most of the light input into photodiode 111A turns into green light while most of the light input into photodiode 112A turns into red light.

As described above, according to living body information sensor 2B in the second modification of the second embodiment, the infrared light included in the light that enters light receiving unit 11 is eliminated by the infrared light elimination layer including infrared cut filter 101 and infrared cut resin 102, so that the measurement accuracy for heart rate measurement can be improved.

Furthermore, since the infrared light elimination layer consists of two layers, further more infrared light can be eliminated as compared with the second embodiment and the first modification of the second embodiment. Consequently, the measurement accuracy for heart rate measurement can be further improved.

### Third Embodiment

In the second embodiment, an explanation has been given with regard to the case where an infrared light elimination layer is used to thereby improve the heart rate measurement accuracy. The oxygen saturation concentration in blood (SpO₂) has conventionally been measured using red light and infrared light. Accordingly, in the second embodiment and the second modification of the second embodiment in which infrared light is eliminated by the infrared light elimination layer, it is difficult to measure the oxygen saturation concentration in blood (SpO₂) by the conventional method. Thus, an explanation will be given in the following with regard to the case where green light is used to thereby allow measurement of the oxygen saturation concentration in blood (SpO₂) in the state where an infrared light elimination layer is still included.

In the third embodiment, the infrared light elimination layer includes an infrared cut filter as in the second embodiment. Furthermore, control device 200 in the second embodiment is replaced with a control device 500. Since other configurations are the same as those in the second embodiment, the description thereof will not be repeated.

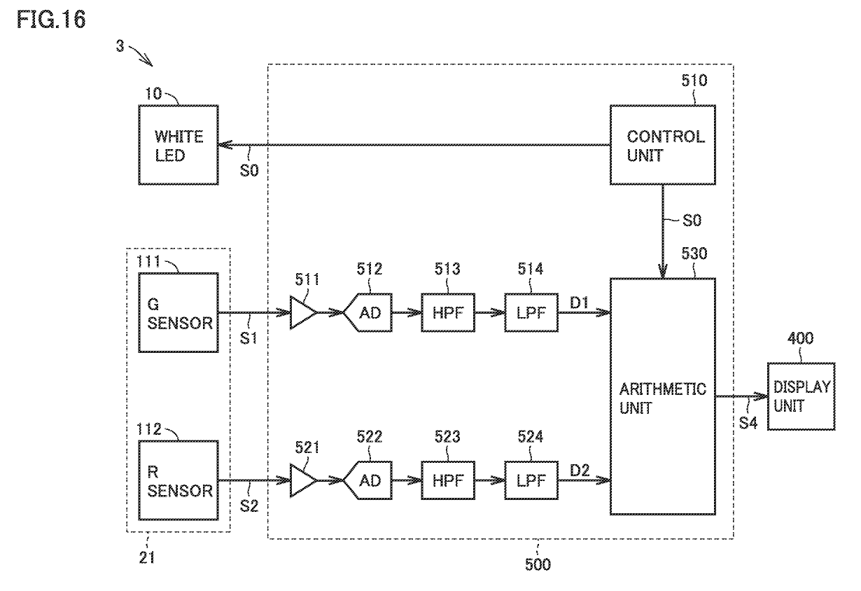

FIG. 16 is a functional configuration diagram of a living body information sensor 3 according to the third embodiment. Referring to FIG. 16, control device 500 includes a control unit 510, amplifiers 511 and 521, AD (Analog-to-Digital) converters 512 and 522, high-pass filters (HPF) 513 and 523, low pass filters (LPF) 514 and 524, and an arithmetic unit 530.

In response to an instruction from the user to start measurement, control unit 510 outputs a signal S0 as a control signal to light emitting unit 10 and arithmetic unit 530. In response to signal S0, arithmetic unit 530 performs an arithmetic operation required for calculating the oxygen saturation concentration in blood.

Amplifier 511 amplifies signal S1 output from G sensor 111. AD converter 512 converts the output signal from amplifier 511 into a digital signal. High-pass filter 513 removes a direct-current (DC) component in the output signal of AD converter 512. Low pass filter 514 removes a noise component of the signal having passed through high-pass filter 513. The signal having passed through low-pass filter 514 is output to arithmetic unit 530 as a digital signal D1.

Amplifier 521 amplifies signal S2 output from R sensor 112. AD converter 522 converts the output signal from amplifier 521 into a digital signal. High-pass filter 523 removes a DC component in the output signal of AD converter 522. Low-pass filter 524 removes a noise component of the signal having passed through high-pass filter 523. The signal having passed through low-pass filter 524 is output to arithmetic unit 530 as a digital signal D2.

Arithmetic unit 530 calculating the oxygen saturation concentration in blood will be hereinafter described. Prior to this description, the method of calculating the oxygen saturation concentration in blood will be first schematically described.

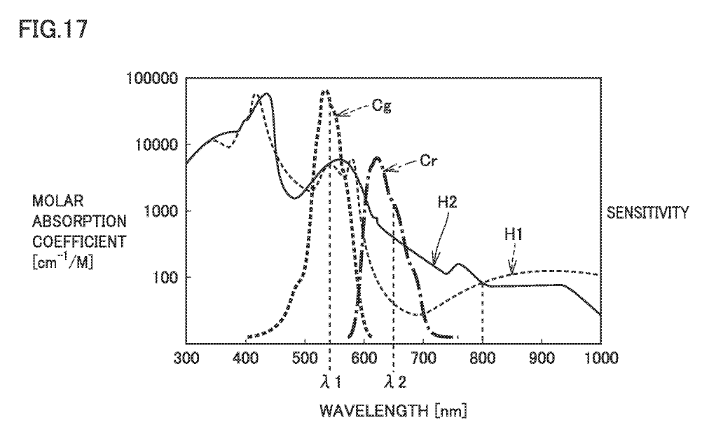

FIG. 17 is a diagram showing the relation among the wavelength [nm] of light, the molar absorption coefficient [cm⁻¹/M] of hemoglobin, the spectral sensitivity of G filter 111B, and the spectral sensitivity of R filter 112B. In FIG. 17, H1 shows a molar absorption coefficient of oxygenated hemoglobin, and H2 shows a molar absorption coefficient of reduced hemoglobin. Also, Cg shows spectral characteristics of G filter 111B, and Cr shows spectral characteristics of R filter 112B. Oxygenated hemoglobin is contained in blood in an artery, and reduced hemoglobin is contained in blood in a vein.

The oxygen saturation concentration in blood shows a proportion of the oxygenated hemoglobin concentration to the sum of the reduced hemoglobin concentration and the oxygenated hemoglobin concentration. Infrared light is likely to absorb more oxygenated hemoglobin than reduced hemoglobin. This means that the molar absorption coefficient of oxygenated hemoglobin to infrared light is greater than the molar absorption coefficient of reduced hemoglobin to infrared light. Red light is likely to absorb more reduced hemoglobin than oxygenated hemoglobin. This means that the molar absorption coefficient of reduced hemoglobin to red light is greater than the molar absorption coefficient of oxygenated hemoglobin to red light. Conventionally, the oxygen saturation concentration in blood has been calculated using the difference between the characteristics of infrared light and red light. Referring to FIG. 17, the molar absorption coefficients of oxygenated hemoglobin and reduced hemoglobin to infrared light in the vicinity of the wavelength of 800 [nm] are almost equal, but the oxygen saturation concentration in blood can be calculated by the conventional calculation method also by using the infrared light having this wavelength. In other words, the oxygen saturation concentration in blood can be calculated by the conventional calculation method as long as light has a wavelength set such that the molar absorption coefficient of oxygenated hemoglobin and the molar absorption coefficient of reduced hemoglobin are equal.

In the third embodiment, since infrared light is eliminated by the infrared light elimination layer including an infrared cut filter, this infrared light cannot be used. In this case, however, the molar absorption coefficients of oxygenated hemoglobin and reduced hemoglobin to green light having a wavelength λ1 in the vicinity of 540 [nm] are almost equal. Thus, in the third embodiment, green light is used in place of infrared light to calculate the oxygen saturation concentration in blood.

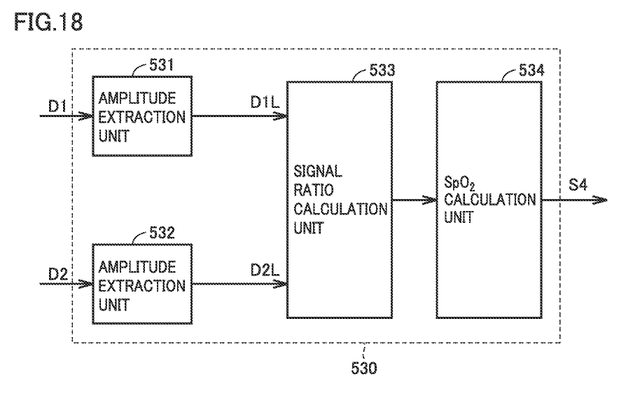

Then, the functional configuration of arithmetic unit 530 will be hereinafter described with reference to FIG. 18. Referring to FIG. 18, arithmetic unit 530 includes amplitude extraction units 531 and 532, a signal ratio calculation unit 533, and an SpO₂ calculation unit 534.

Amplitude extraction unit 531 extracts an amplitude value of the intensity of green light from a digital signal D1 received from G sensor 111, and outputs the extracted value as a digital signal D1L to signal ratio calculation unit 533. Amplitude extraction unit 532 extracts an amplitude value of the intensity of red light from a digital signal D2 received from R sensor 112, and outputs the extracted value as a digital signal D2L to signal ratio calculation unit 533. When amplitude extraction units 531 and 532 each extract the amplitude value of the intensity of light from the digital signal, the amplitude value may be amplified at a prescribed ratio. Signal ratio calculation unit 533 calculates a ratio R between a voltage value V1 of digital signal D1L and a voltage value V2 of digital signal D2L (=V1/V2), and outputs the calculated ratio R to SpO₂ calculation unit 534. The less the ratio between voltage value V1 of digital signal D1L and voltage value V2 of digital signal D2L is, the greater the oxygen saturation concentration in blood is.

Based on the previously-prepared correspondence table showing the corresponding relation between ratio R and the oxygen saturation concentration in blood, SpO₂ calculation unit 534 calculates the oxygen saturation concentration in blood and outputs the calculated concentration to display unit 400. This correspondence table can be obtained in advance by real machine experiments or simulations.

As described above, living body information sensor 3 according to the third embodiment allows measurement of the oxygen saturation concentration in blood while an infrared light elimination layer is still provided. In other words, according to living body information sensor 3, the oxygen saturation concentration in blood can also be measured while infrared light acting as noise when measuring a heart rate is eliminated by the infrared light elimination layer so as to improve the measurement accuracy for heart rate measurement.

In the third embodiment, an explanation has been given with regard to the case where the infrared light elimination layer includes an infrared cut filter. The infrared light elimination layer used when measuring the oxygen saturation concentration in blood does not necessarily have to include an infrared cut filter. The infrared light elimination layer used when measuring the oxygen saturation concentration in blood can include, for example, an infrared cut resin as in the first modification of the second embodiment, or can include an infrared cut filter and an infrared cut resin as in the second modification of the second embodiment.

Light emitting unit 10 emitting white light is used in the first to third embodiments. However, since only green light and red light of white light are used, a green LED that emits green light and a red LED that emits red light may be provided in place of light emitting unit 10.

Although one light emitting unit 10 and one light receiving unit 11 are used in each of the first to third embodiments, a plurality of light emitting units 10 and one light receiving unit 11 may be used, in which case this one light receiving unit 11 may be arranged in the center of the plurality of light emitting units 10.

In the second and third embodiments, the infrared light elimination layer is bonded to silicon substrate 12. The infrared light elimination layer does not necessarily have to be bonded to silicon substrate 12, but may be formed anywhere as long as it is located on the optical path leading to photodiode 111A and also on the optical path leading to photodiode 112A. For example, the infrared light elimination layer may be formed inside each of G sensor 111 and R sensor 112 serving as optical sensors, or may be formed at a position away from silicon substrate 12.

The infrared light elimination layer can include not only an infrared cut filter and an infrared cut resin, but also include any element as long as it can eliminate infrared light.

Explanations have been given in the second embodiment, the first modification of the second embodiment and the third embodiment with regard to the case where a single infrared light elimination layer is provided, and also given in the second modification of the second embodiment with regard to the case where two infrared light elimination layers are provided. The number of infrared light elimination layers is not limited to the above, but may be three or more.

In the second and third embodiments, the infrared light elimination layer eliminates infrared light from the light having entered the light receiving unit. It can be recognized that infrared light is eliminated, for example, not only by the infrared light elimination layer including an infrared cut filter or an infrared cut resin, but also by the color filter through which light having a prescribed wavelength is allowed to pass. It can also be recognized that the infrared light elimination layer in the present invention further enhances the effect of infrared light elimination, which can be achieved also by the color filter.

The living body information sensor according to each of the first to third embodiments allows detection of the heart rate or the oxygen saturation concentration in blood not only of a human body but also of living bodies including humans and animals.

The embodiments disclosed herein are intended to be combined as appropriate. It should be construed that embodiments disclosed herein are by way of illustration in all respects, not by way of limitation. The scope of the present invention is defined by the terms of the claims, rather than the description of the embodiments provided above, and is intended to include any modifications within the meaning and scope equivalent to the terms of the claims.

What is claimed is:

1. A living body information sensor attachable to a living body, the living body information sensor comprising:
a light emitting unit emitting light to the living body;
a light receiving unit receiving reflected light from the living body that is included in the light emitted by the light emitting unit to the living body, and outputting a signal according to intensity of the reflected light; and
a control device generating information about the living body based on the signal from the light receiving unit,
the control device
controlling the light emitting unit to repeatedly turn on and off in an alternate manner, and
setting a total of a turn-on time of the light emitting unit in a prescribed time interval to be smaller when the living body information sensor is attached to the living body than when the living body information sensor is not attached to the living body.

2. The living body information sensor according to claim 1, wherein the control device sets a turn-on interval of the light emitting unit to be longer when the living body information sensor is attached to the living body than when the living body information sensor is not attached to the living body.

3. The living body information sensor according to claim 2, wherein the control device sets the turn-on interval based on a first signal received from the light receiving unit in a state where the light emitting unit turns on and a second signal received from the light receiving unit in a state where the light emitting unit turns off

4. The living body information sensor according to claim 3, wherein
the control device
calculates a difference obtained by subtracting a level of the second signal from a level of the first signal,
sets the turn-on interval at a first turn-on interval when the difference is smaller than a prescribed threshold value, and
sets the turn-on interval at a second turn-on interval longer than the first turn-on interval when the difference is larger than the prescribed threshold value.

5. The living body information sensor according to claim 1, wherein
the control device
controls the light emitting unit to repeatedly turn on and off in an alternate manner in a prescribed turn-on period, and
sets a ratio of the turn-on time of the light emitting unit to the prescribed turn-on period to be less when the living body information sensor is attached to the living body than when the living body information sensor is not attached to the living body.

6. The living body information sensor according to claim 1, further comprising:
a substrate on which the light emitting unit, the light receiving unit and the control device are formed;
a first light shielding wall disposed to surround the light emitting unit along an outer surrounding edge of the substrate; and
a second light shielding wall partitioning space provided by the substrate and the first light shielding wall into space in which the light emitting unit is located and space in which the light receiving unit is located.

7. A living body information sensor including a light receiving unit and generating information about a living body based on a signal from the light receiving unit,
the light receiving unit including a first optical sensor converting first light into a first signal,
the first optical sensor including
a first photoelectric conversion element converting the first light into the first signal, and
a first color filter formed on an optical path leading to the first photoelectric conversion element, the first light passing through the first color filter, and
the living body information sensor further comprising an infrared light elimination layer formed on the optical path leading to the first photoelectric conversion element.

8. The living body information sensor according to claim 7, further comprising a control device generating the information about the living body based on the signal from the light receiving unit, wherein
the light receiving unit further includes a second optical sensor converting second light into a second signal,
the second optical sensor includes
a second photoelectric conversion element converting the second light into the second signal, and
a second color filter formed on an optical path leading to the second photoelectric conversion element, the second light passing through the second color filter,
the infrared light elimination layer is formed also on the optical path leading to the second photoelectric conversion element, and
the control device receives the first signal and the second signal from the light receiving unit, and generates the information about the living body based on the first signal and the second signal.

9. The living body information sensor according to claim 8, wherein
the light receiving unit further includes a first substrate on which the first optical sensor and the second optical sensor are formed, and
the infrared light elimination layer is formed on a surface of the first substrate.

10. The living body information sensor according to claim 9, wherein
the infrared light elimination layer includes an infrared cut filter, and
the infrared cut filter covers the first color filter and the second color filter.

11. The living body information sensor according to claim 9, wherein
the infrared light elimination layer includes an infrared cut resin, and
the infrared cut resin covers the first substrate.

12. The living body information sensor according to claim 9, wherein
the infrared light elimination layer includes an infrared cut filter and an infrared cut resin,
the infrared cut filter covers the first color filter and the second color filter, and
the infrared cut resin is stacked on the infrared cut filter, and covers the first substrate.

13. The living body information sensor according to claim 8, further comprising:
a light emitting unit emitting light including the first light and the second light;
a second substrate on which the light emitting unit, the light receiving unit and the control device are formed;
a first light shielding wall formed to surround the light emitting unit and the light receiving unit along an outer surrounding edge of the second substrate; and
a second light shielding wall partitioning space provided by the second substrate and the first light shielding wall into space in which the light emitting unit is located and space in which the light receiving unit is located, wherein
when the living body information sensor is attached to the living body, light included in the light and reflected inside the living body enters the light receiving unit.

14. The living body information sensor according to claim 13, wherein the light emitting unit is a light emitting diode that emits white light.

15. The living body information sensor according to claim 8, wherein
the first light is green light, and
the second light is red light.

16. The living body information sensor according to claim 8, wherein the control device generates the information about the living body based on a difference between a level of the first signal and a level of the second signal.

17. The living body information sensor according to claim 16, wherein the information about the living body is a heart rate.

18. The living body information sensor according to claim 8, wherein the control device generates the information about the living body based on a ratio between a level of the first signal and a level of the second signal.

19. The living body information sensor according to claim 18, wherein the information about the living body is an oxygen saturation concentration in blood.
